(19) United States
(12) Patent Application Publication
(10) Pub. No.: US 2006/0246507 A1
(43) Pub. Date: Nov. 2, 2006

(54) Beta-arrestin based screening assays

(75) Inventor: Anders Heding, Copenhagen O (DK)

Correspondence Address: EDWARDS & ANGELL, LLP, P.O. BOX 55874, BOSTON, MA, 02205, US

(73) Assignee: 7TM Pharma A/S, Horsholm (DK)

(21) Appl. No.: 10/543,349

(22) PCT Filed: Jan. 20, 2004
(86) PCT No.: PCT/DK04/00033

§ 371 Date: May 12, 2006

(30) Foreign Application Priority Data
Jan. 22, 2003 (DK) PA 2003 00078

(51) Int. Cl. G01N 33/53; C12Q 1/37

(52) U.S. Cl. 435/7.1; 435/23

(57) ABSTRACT

Use of mutated β-arrestin for an improved enzyme complementation assay or translocation assay, the improved enzyme complementation assay comprising: i) adding a substrate to a cell comprising a GPCR-EA fusion protein and a β-arrestin-EB fusion protein, wherein the β-arrestin is mutated, ii) adding a ligand to obtain, if possible, a GPCR-EA/β-arrestin-EB complex, and iii) measuring a signal arising from association of EA and EB to create an enzymatically active protein catalyzing conversion of the substrate which leads to a detectable signal, wherein the improvement leads to an increased signal compared with the signal obtained by use of the same process employing a β-arrestin-EB fusion protein, wherein the β-arrestin is wild type β-arrestin, and the improved β-arrestin translocation assay comprising i) providing a cell expressing a GPCR and comprising a β-arrestin associated with an optically detectable molecule, ODM, wherein the β-arrestin is mutated, ii) adding a ligand to obtain, if possible, a GPCR/β-arrestin complex, and iii) detecting a translocation of the optically detectable molecule, wherein the improvement leads to a increased and prolonged translocation of the β-arrestin associated with an optically detectable molecule as compared with the signal obtained by use of the same assay employing a β-arrestin associated with an optically detectable molecule, wherein the β-arrestin is wild type β-arrestin.

### FIELD OF THE INVENTION

The present invention describes an improved enzyme complementation assay, wherein the enzyme-generated signal is enhanced and/or prolonged. The improved enzyme complementation assay comprises the following steps:

i) adding a substrate to a cell comprising a GPCR fused to a non-functional part of an enzyme or protein (GPCR-EA) and a β-arrestin fused to another non-functional part of the enzyme or protein (β-arrestin-EB), wherein the β arrestin is mutated,

ii) adding a ligand to obtain, if possible, a GPCR-EA/β-arrestin-EB complex, and

iii) measuring a signal arising from association of EA and EB to create an enzymatically active protein catalyzing conversion of a substrate which leads to a detectable signal.

The improvement leads to an increased signal compared with the signal obtained by use of the same process employing a β-arrestin-EB fusion protein, wherein the β-arrestin is wild type β-arrestin

The present invention also describes an improved β-arrestin translocation assay, wherein the β-arrestin translocation is enhanced and/or prolonged. The improved β-arrestin translocation assay comprises the following steps:

i) providing a cell expressing a GPCR and comprising a β-arrestin associated with an optically detectable molecule, ODM, wherein the β arrestin is mutated,

ii) adding a ligand to obtain, if possible, a GPCR/β-arrestin complex, and

iii) detecting a translocation of the optically detectable molecule.

The improvement leads to an increased and prolonged translocation of the β-arrestin associated with an optically detectable molecule as compared with the signal obtained by use of the same assay employing a β-arrestin associated with an optically detectable molecule, wherein the β-arrestin is wild type β-arrestin.

### BACKGROUND OF THE INVENTION

Enzyme Complementation Assay

The enzyme complementation assay is a protein-protein interaction assay. The assay involves a receptor fused to a part of an enzyme or protein where the enzyme part is not functional, and a β-arrestin fused to another non-functional part of the enzyme or protein.

When the receptor is activated the receptor will bind the β-arrestin molecule. This brings the two parts of the enzyme in close proximity and they will now combine into a functional enzyme. The enzyme can now process its substrate and this can be measured as e.g., light output or color change.

A specific example is the use of the enzyme beta-galactosidase (β-gal). An example of two complementary parts of this enzyme is an N-terminal deletion (Δα) and a C-terminal deletion (Δω) (Journal of Biomolecular Screening, vol. 6, number 6, pp. 401-411 and Journal of Biomolecular Screening, vol. 7, number 5, pp. 451-459). If for example the Δα β-gal fragment is fused to a GPCR (7TM receptor) and the Δω β-gal fragment is fused to β-arrestin then the two by them self's inactive fragments will be brought together to form an active enzyme when the receptor/β-arrestin complex is formed. The now active enzyme will now be able to catalyze a substrate for example Gal-Screen® Reagent, and the enzymatic activity can be measured as light output.

However, in certain situations where a GPCR-arrestin based assay is used, the enzymatic signal is relatively weak and short termed. Thus, there is a need for improving the enzyme complementation assay in order to obtain a prolonged and/or enhanced signal.

β-Arrestin Translocation Assay

The β-arrestin translocation assay is a protein-protein interaction assay. This assay involves a cell expressing a GPCR and a β-arrestin fused to e.g. a fluorescent molecule, such as a GFP molecule. The receptors are located in the membrane of the cell, and the β-arrestin molecules are located in the cytoplasm. When the receptor is activated by an agonist the receptor will bind the β-arrestin fusion molecule. This can be seen as a movement (translocation) of fluorescence, if a fluorescent molecule is used, from the cytoplasm to the membrane or just as a general movement of fluorescence (Assay and Drug Development Technologies, Vol. 1, number 1, 2002, pp. 21-30).

However, in certain situations where a GPCR-arrestin based assay is used, the β-arrestin translocation is relatively weak and short termed. Thus, there is a need for improving the β-arrestin translocation assay in order to obtain a prolonged and/or enhanced β-arrestin translocation.

### DETAILED DISCLOSURE OF THE INVENTION

Accordingly, the present invention provides an improved enzyme complementation assay comprising the following steps:

i) adding a substrate to a cell comprising a GPCR fused to a non-functional part of an enzyme or protein, GPCR-EA, and a β-arrestin fused to another non-functional part of the enzyme or protein, β-arrestin-EB, wherein the β-arrestin is mutated,

ii) adding a ligand to obtain, if possible, a GPCR-EA/β-arrestin-EB complex, and

iii) measuring a signal arising from association of EA and EB to create an enzymatically active protein catalyzing conversion of a substrate which leads to a detectable signal.

The improvement leads to an increased signal compared with the signal obtained by use of the same process employing a β-arrestin-EB fusion protein, wherein the β-arrestin is wild type β-arrestin.

The present invention also provides an improved β-arrestin translocation assay comprising the following steps:

i) providing a cell expressing a GPCR and comprising a β-arrestin associated with an optically detectable molecule, ODM, wherein the β arrestin is mutated,

ii) adding a ligand to obtain, if possible, a GPCR/β-arrestin complex, and

iii) detecting a translocation of the optically detectable molecule.

The improvement leads to a increased and prolonged translocation of the β-arrestin associated with an optically detectable molecule as compared with the signal obtained by use of the same assay employing a β-arrestin associated with an optically detectable molecule, wherein the β-arrestin is wild type β-arrestin.

G Protein-Coupled Receptors for Use in the Present Invention

The G protein-coupled receptors (GPCRs) constitute the largest family of proteins in the human genome and function as receivers of all kinds of chemical signals. The spectrum of hormones, neurotransmitters, paracrine mediators etc., which act through G-protein coupled receptors includes all kinds of chemical messengers: Ions (calcium ions acting on the parathyroid and kidney chemosensor), amino acids (glutamate and -amino butyric acid —GABA), monoamines (catecholamines, acetylcholine, serotonin, etc.), lipid messengers (prostaglandins, thromboxane, anandamide, (endogenous cannabinoid), platelet activating factor, etc.), purines (adenosine and ATP), neuropeptides (tachykinins, neuropeptide Y, endogenous opioids, cholecystokinin, vasoactive intestinal polypeptide (VIP), plus many others), peptide hormones (angiotensin, bradykinin, glucagon, calcitonin, parathyroid hormone, etc.), chemokines (interleukin-8, RANTES, MIP-1alpha etc.), glycoprotein hormones (TSH, LH/FSH, choriongonadotropin, etc.), as well as proteases (thrombin). In our sensory systems, G-protein coupled receptors are involved both as the light sensing molecules in the eye, i.e. rhodopsin and the color pigment proteins, and as several hundreds of distinct odorant receptors in the olfactory system as well as a large number of taste receptors. Structurally, G protein coupled receptors (GPCRs) are characterized by seven hydrophobic helical transmembrane segments connected by intra- and extracellular loops and are accordingly often referred to as 7TM receptors.

Examples of 7TM receptors are the receptors for (—in brachet the receptor subtypes are mentioned): acetylcholine (m1-5), adenosine (A1-3) and other purines and purimidines (P2U and P2Y1-12), adrenalin and noradrenalin (α1A-D, α2A-D and β1-3), amylin, adrenomedullin, anaphylatoxin chemotactic factor, angiotensin (AT1A, -1B and -2), apelin, bombesin, bradykinin (1 and 2), C3a, C5a, calcitonin, calcitonin gene related peptide, CD97, conopressin, corticotropin releasing factor (CRF1 and -2), calcium, cannabinoid (CB1 and -2), chemokines (CCR1-11, CXCR1-6, CX3CR and XCR), cholecystokinin (A-B), corticotropin-releasing factor (CRF1-2), dopamine (D1-5), eicosanoids, endothelin (A and B), fMLP, Frizzled (Fz1,2,4,5 and 7-9), GABA(B1 and B2), galanin, gastrin, gastric inhibitory peptide, glucagon, glucagon-like peptide I and II, glutamate (1-8), glycoprotein hormone (e.g. FSH, LSH, TSH, LH), growth hormone releasing hormone, growth hormone secretagogue/Ghrelin, histamine (H1-4), 5-hydroxytryptamine (5HT1A-1F, -2A-C and -4-7), leukotriene, lysophospholipid (EDG1-4), melanocortins (MC1-5), melanin concentrating hormone (MCH 1 and 2), melatonin (ML1A and 1B), motilin, neuromedin U, neuropeptide FF (NFF1 and 2), neuropeptide Y (NPY1,2,4,5 and 6), neurotensin (1 and 2), nocioceptin, odor components, opiods (κ, δ, μ and x), orexins(OX1 and -2), oxytocin, parathyroid hormone/parathyroid hormone-related peptides, pheromones, platelet-activating factor, prostaglandin (EP1-4 and F2) prostacyclin, pituitary adenylate activating peptide, retinal, secretin, smoothernd, somatostatins (SSTR1-5), tachykinins (NK1-3), thrombin and other proteases acting through 7TM receptor, thromboxane, thyrotropin-releasing hormone, vasopressin (V1A, -1B and -2), vasoactive intestinal peptide, urotensin II, and virally encoded receptors (US27, US28, UL33, UL78, ORF74, U12, U51); and 7TM proteins coded for in the human genome but for which no endogenous ligand has yet been assigned such as mas-proto-oncogene, EBI (I and II), lactrophilin, brain specific angiogenesis inhibitor (BAI1-3), EMR1, RDC1 receptor, GPR12 receptor or GPR3 receptor, and 7TM proteins coded for in the human genome but for which no endogenous ligand has yet been assigned.

Arrestins Role in Receptor Signalling

Arrestins play an important role in the regulation of 7TM receptor responsiveness by terminating the G protein mediated signal. Arrestins are cytosolic proteins, which upon agonist binding to 7TM receptors are translocated to the activated and usually phosphorylated receptor within seconds or minutes after agonist stimulation. Full inactivation of 7TM receptor signaling is achieved through binding of one of a family of arrestin molecules, which sterically hinder G protein binding.

Arrestin functions as an adaptor protein, which will connect the receptor to clathrin and AP-2 in clathrin coated pits, which results in sequestration of the receptor into intracellular vesicles of the endosomal pathway in which dynamin plays an important role in the actual vesicular sequestration process. The mechanisms involved in the transport of the arrestin-receptor complex to the clathrin coated pit is not fully understood, but it is becoming clear that the binding of arrestin to parts of the cell membrane e.g. to phosphoinositides is essential.

The family of arrestins has at least four members showing a high degree of amino acid homology and classified primarily on the basis of tissue distribution. They include (i) visual arrestin and (ii) C-arrestin, which are mostly restricted to the eye, and the non-visual-arrestins (iii) β-arrestin1 and (iv) β-arrestin 2, distributed ubiquitously in almost every tissue. β-arrestins share more than 70% amino acid identity.

Arrestins are composed of three structural and functional parts, an amino-terminal domain, which binds to the receptor, a carboxyl-terminal domain, which connects to proteins involved in receptor-sequestration, such as clathrin and AP-2 (adaptor protein 2) and a central part which connects to components of the cell membrane, such as phosphoinositides. Visual arrestins, which mainly interacts with the rhodopsin receptor, are very weak in their clathrin-association [Goodman, 1996, Nature] and are in general not considered to be capable of mediating receptor internalisation.

As described above arrestin are translocated to the activated and usually phosphorylated GPCR within minutes after agonist stimulation. This interaction is universal for almost all GPCR's upon activation. Thus, an enzyme complementation assay or a translocation assay based on the GPCR-arrestin interaction, is very useful assays for a wide range of receptors, and also provides means for the discovery of ligands that interact with GPCRs of unknown function i.e. orphan GPCRs.

However, as described above, arrestin functions as an adaptor protein, which will connect he receptor to clathrin and AP-2, which results in sequestration of the receptor into intracellular vesicles. After internalization of the receptor/arrestin complex, arrestin will dissociate from the receptor and the enzymatic signal will be terminated if an enzyme complementation assay were performed. If a β-arrestin translocation assay were performed the translocation would be terminated. The dissociation kinetics can be fast or slow depending on the receptor type. For Class A type receptors, the dissociation is usually fast, whereas for Class B type receptors the dissociation is slower.

The present invention describes an improved enzyme complementation assay and an improved β-arrestin translocation assay wherein the generated signals are enhanced and/or prolonged.

Definitions

In the present context the term “EA” is intended to illustrate a non-functional part of a protein or enzyme. The term “EB” is intended to illustrate another non-functional part of the protein or enzyme. The combination of “EA” and “EB” will provide an enzymatically functional protein. In certain cases fusion of EA and EB provides an enzyme.

A “ligand” is intended to include a substance that either inhibits or stimulates the activity of a receptor and/or that competes for the receptor in a binding assay. An “agonist” is defined as a ligand increasing the functional activity of a biological target molecule. An “antagonist” is defined as a ligand decreasing the functional activity of a biological target molecule either by inhibiting the action of an agonist or by its own intrinsic activity. An “inverse agonist” (also termed “negative antagonist”) is defined as a ligand decreasing the basal functional activity of a biological target molecule

In the present context the term “improved enzyme complementation assay” denotes an assay where the generated signal is increased by at least about 5% such as, e.g., at least about 10%, at least about 15%, at least about 20%, at least about 25%, at least about 30%, at least about 35%, at least about 40%, at least about 45%, at least about 50%, at least about 55%, at least about 65%, at least about 70%, at least about 75%, at least about 80%, or more.

In the present context the term “improved β-arrestin translocation assay” denotes an assay where the translocation is increased and prolonged by at least about 5% such as, e.g., at least about 10%, at least about 15%, at least about 20%, at least about 25%, at least about 30%, at least about 35%, at least about 40%, at least about 45%, at least about 50%, at least about 55%, at least about 65%, at least about 70%, at least about 75%, at least about 80%, or more.

In the present context, the term “β-arrestin that is specifically mutated so that it acts on the receptor independent of the receptors phosphorylation state” means that β-arrestin is mutated with the purpose of becoming phosphorylation independent. An example of such a mutation is R169E human β-arrestin-2, wherein arginine has been changed to glutamic acid.

Since the signal generated from the enzyme complementation assay is dependent on the association/dissociation of the GPCR-EA/β-arrestin-EB complex, prevention of the dissociation of the complex will enhance and/or prolong the signal.

The same applies for the β-arrestin translocation assay, where the signal is dependent on the association/dissociation of the GPCR/β-arrestin-ODM complex, and where prevention of the dissociation of the complex will also enhance and/or prolong the signal.

Thus, the invention provides improved assays, wherein the separation of β-arrestin from either the GPCR-EA/β-arrestin-EB or the GPCR/β-arrestin-ODM complex is delayed and/or inhibited.

As described above, the GPCR/β-arrestin complex dissociates when the complex is internalized. Thus, inhibition of the internalization will prevent dissociation and accordingly, the generated signals will be enhanced and/or prolonged. Accordingly, the invention also relates to an improved enzyme complementation assay wherein the internalization of the GPCR-EA/β-arrestin-EB complex is inhibited. The invention further relates to an improved translocation assay wherein the internalization of the GPCR/β-arrestin-ODM complex is inhibited.

One way of inhibiting internalization is to prevent the binding of β-arrestin to clathrin and AP-2. In improved enzyme complementation and translocation assays according to the invention, β-arrestin is mutated so that its binding to clathrin and/or AP2 is impaired.

The β-arrestin may further be mutated so that it is phosphorylation independent, i.e. it will act on a receptor independent of the receptors phosphorylation state.

As described above, the arrestins are composed of structural and functional parts, an amino-terminal domain, which binds to the receptor and a carboxyl-terminal domain, which connects to proteins involved in receptor-sequestration, such as clathrin and AP-2. Accordingly, in some assays according to the invention, the β-arrestin is truncated so that it does not contain any clathrin and/or AP2 binding sites, i.e. a part of the β-arrestin from the C-terminal end has been deleted.

In other assays according to the invention β-arrestin is mutated by deletion, insertion or substitution so that one or more AP2 binding sites of β-arrestin are impaired in their binding to AP2.

Specific examples of a truncated β-arrestin are a human β-arrestin 1 374 stop mutant or human β-arrestin 2 373 stop mutant.

A specific example of a β-arrestin mutated by substitution is the human β-arrestin 2 R393E;R395E mutant, wherein the amino acids number 393 and 395 have been changed from arginine to glutamic acid. Another example of a β-arrestin mutated by substitution is the human β-arrestin-2 R393A;R395A mutant, wherein the amino acids number 393 and 395 have been changed from arginine to alanine. Other substitutions may of course be used.

As described above, there are at least 4 family members of arresting. Furthermore, the arrestins are found in animals including rodents, swine, poultry, cattle, sheep, goats, horses, cats, dogs, monkeys and humans. Thus, the specific mutations mentioned only tend as illustrative examples. All arrestins are usable in assays according to the invention, and the specific position of the truncation and other mutations will depend on the species and type of arrestin. As an example, to impair the AP2 binding sites in bovine β-arrestin 2 amino acids number 394 and 396 should be substituted from arginine to glutamic acid as compared to the R393E and R395E substitutions in human β-arrestin.

Another way of inhibiting the internalization is to prevent the binding of β-arrestin to phosphoinositide. By impairing the binding of β-arrestin to phosphoinositide, the transport to the clathrin coated pits is impaired.

In order to impair binding to phosphoinositide β-arrestin may be mutated in any suitable way e.g. by deletion, insertion or substitution.

A specific example of a β-arrestin mutant, wherein the binding to phosphoinositide is impaired is the triple mutant human β-arrestin-2 K233Q;R237Q;K251Q, wherein amino acid no. 233 has been changed from lysine to glutamine, amino acid no. 237 has been changed from arginine to glutamine and amino acid no. 251 has been changed from lysine to glutamine.

In an improved enzyme complementation assay according to the invention the non-functional enzyme part, EA, may be beta-galactosidase comprising a N-terminal, Δα, deletion, and the other non-functional part of the enzyme, EB, may be beta-galactosidase comprising a C-terminal deletion, Δω.

In another enzyme complementation assay according to the invention the enzymatically active protein may be lactosidase, and EA and EB may be non-functional parts of lactosidase.

The use of beta-galactosidase and lactosidase only tends to illustrate the invention. All enzymes or proteins catalyzing a process that may be visualized by e.g. color change, light emission etc may be used.

The invention also provides an improved translocation assay, wherein the optically detectable molecule may be a fluorescent molecule, such as, e.g. a GFP molecule. In another translocation assay according to the invention the optically detectable molecule may be a luminescent molecule.

### APPLICATIONS OF THE INVENTION

The improved assays may be used in drug discovery methods, such as screening assays for identifying new ligands of GCPRs. The assays may also be used for the discovery of ligands that interact with GPCRs of unknown function, i.e. orphan GPCRs.

The ligands may be agonists or antagonists. If the ligand is a known antagonist, or if the assay is set up to screen for unknown antagonists, the improved assays further comprises the addition of an agonist after adding the antagonist, or suspected antagonist ligand.

The invention also relates to improved assays according to the invention for use in high-throughput screening.

### OTHER ASPECTS OF THE INVENTION

Other aspects of the invention appear from the appended claims. The details and particulars described above and relating to the methods according to the invention apply mutatis mutandis to the other aspects of the invention.

### LEGENDS TO FIGURES

FIG. 1 shows internalization of the NK1 and the P2AR receptors co-expressed in cells together with WT or one of the three different β-arrestin mutants: human β-arrestin-2 R393E;R395E mutant, human β-arrestin-2 373 stop mutant or human β-arrestin-2 R169E mutant.

The following examples are intended to illustrate the invention without limiting it thereto.

### EXAMPLES

NK-1 Receptor Internalization Assays

COS-7 cells in 75 cm² flask (3×10⁶ cells/flask) were used for transfection. NK-1/Rluc receptor (2 μg cDNA/flask) was coexpressed together with 6 μg GFP²/β-arrestin 2, 6 μg GFP²/β-arrestin R169E, 6 μg GFP²/β-arrestin Lys 373 stop or 6 μg GFP²/β-arrestin R393E, R395E. At the end of transfection period (3-5 hours), cells were washed twice with PBS, trypsinased and plated at a density of 2.5×10⁵ cells per well in 12-well plates. After 48 hours, cells were washed once with assay medium (HEPES-modified DMEM with 0.1% BSA, pH 7.4) and incubated in assay medium for at least 1 hour before being incubated with ¹²⁵I-labeled SP (30000 cpm/well) in 0.5 ml assay medium 10 min at 37 C. Cells were then transferred onto ice and washed twice with ice-cold PBS. Subsequently, the extracellular receptor-associated ligand was removed by washing once with 1 ml of acid solution (50 mM acetic acid and 150 mM NaCl, pH 2.8) for 12 min. The acid wash was collected to determine the surface-bound radioactivity, and the internalized radioactivity was determined after solubilizing the cells in 0.2 M NaOH and 1% SDS NaOH/SDS) solution. Nonspecific binding for each time point was determined under the same conditions in the presence of 1 μM unlabeled agonist (SP). After subtraction of nonspecific binding, the internalized radioactivity was expressed as a percentage of the total binding.

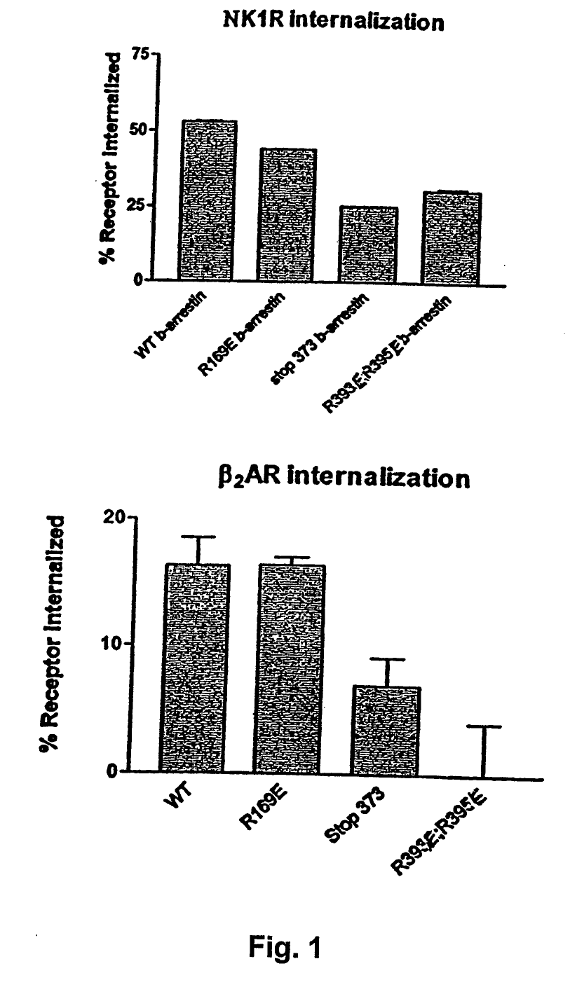

FIG. 1 shows the internalization of the NK1-R co-expressed in cells together with WT or one of the three different β-arrestin mutants. The figure illustrates that the human β-arrestin-2 R393E;R395E mutant and the human β-arrestin-2 373 stop mutant are inhibiting the internalization.

β₂AR Internalization Assays

COS-7 cells in 75 cm² flask (3×10⁶ cells/flask) were used for trasfection. β₂AR/Rluc receptor (1.3 μg cDNA/flask) was coexpressed together with 6.5 μg GFP²/β-arrestin 2, 6.5 μg GFP²/β-arrestin R169E, 6.5 μg GFP²/β-arrestin Lys 373 stop or 6.5 μg GFP²/β-arrestin R393E, R395E. Receptor internalization assay was based on protocol described by Barak and Caron J Recept Signal Transduct Res 1995 January-March; 15(1-4):677-90. At the end of transfection period (3-5 hours), cells were washed twice with PBS, trypsinesed and plated at a density of 2.5×10⁵ cells per well in 12-well plates. After 48 hours, cells were washed once with assay medium (HEPES-modified DMEM with 0.1% BSA, pH 7.4) and serum-starved in the same medium for additional 2-3 hours before being stimulated with 1 mM isopterenol for 10 min at 37° C. Stimulation was stopped by washing the cells with ice-cold PBS. Cells were then subjected to [¹²⁵I]-pindolol binding at 4° C. for 3 h and the fraction of internalized receptors determined relative to unstimulated cells. Non-specific binding was determined under the same conditions in the presence of 1 μM pindolol.

FIG. 1 shows the internalization of the β2AR co-expressed in cells together with Wt or one of the three different β-arrestin mutants. The figure illustrates that the human β-arrestin-2 R393E;R395E mutant and the human β-arrestin-2 373 stop mutant are inhibiting the internalization.

It is also shown that the effect is most significant for the β2AR receptor as compared to the NK1 receptor.

Enzyme complementation Assay

Cell Work

COS-7 cells in 75 cm² flask (3×10⁶ cells/flask) were used for transfection. β₂AR/Δα receptor (1.3 μg cDNA/flask) was coexpressed together with 6.5 μg β-arrestin/Δω, 6.5 μg β-arrestin/Δω R169E, 6.5 μg β-arrestin/Δω Lys 373 stop or 6.5 μg β-arrestin/Δω R393E, R395E. At the end of transfection period (3-5 hours), cells were washed twice with PBS, trypsinesed and plated out as described below.

Agonist
1. Seed cells expressing the β₂-Adrenergic receptor construct and a β-arrestin constructs (1×10⁴ cells/well) in a 96 well plate
2. Culture for 24 h
3. Remove media
4. Add 90 μl fresh media/well
5. Add compounds (10 μl/well to 10 μM)
6. Incubate for 60 min
7. Remove media and compound
8. Add Gal-Screen® reagent
9. Incubate for 60 min
10. Measure light emission

Antagonist:
1. Seed cells expressing the β₂-Adrenergic receptor construct and a β-arrestin constructs (1×10⁴ cells/well) in a 96 well plate
2. Culture for 24 h
3. Remove media
4. Add 80 μl fresh media/well
5. Add compounds (10 μl/well to 101 μM)
6. Add isoproterenol (10 μl/well to 50 nM)
7. Incubate for 60 min
8. Remove media and compound
9. Add Gal-Screen® reagent
10. Incubate for 60 min
11. Measure light emission

β-Arrestin Translocation Assay

Cell Work

COS-7 cells in 75 cm² flask (3×10⁶ cells/flask) were used for transfection. β₂AR receptor (1.3 μg cDNA/flask) was co-expressed together with 6.5 μg β-arrestin/GFP, 6.5 μg β-arrestin/GFP R169E, 6.5 μg β-arrestin/GFP Lys 373 stop or 6.5 μg β-arrestin/GFP R393E, R395E. At the end of the transfection period (3-5 hours), cells were washed twice with PBS, trypsinesed and plated out as described below.

Agonist
1. Seed cells expressing the β₂-Adrenergic receptor and a β-arrestin constructs (1.5×10⁴ cells/well) in a 96 well plate
2. Culture for 24 h
3. Add compounds (10 μl/well to 10 μM)
4. Incubate for 30 min
5. Fix cells with 2% paraformaldehyde for 1 h
6. Remove paraformaldehyde
7. Stain nucleus with Hoechst (1 μg/ml)
8. Measure β-arrestin translocation using appropriate reader and data analysis algoritm

Antagonist
1. Seed cells expressing the β₂-Adrenergic receptor and a β-arrestin constructs (1.5×10⁴ cells/well) in a 96 well plate
2. Culture for 24 h
3. Add compounds (10 μl/well to 10 μM)
4. Incubate for 30 min
5. Add isoproterenol (10 μl/well to 50 nM
6. Incubate for 30 min
7. Fix cells with 2% paraformaldehyde for 1 h
8. Remove paraformaldehyde
9. Stain nucleus with Hoechst (1 μg/ml)
10. Measure β-arrestin translocation using appropriate reader and data analysis algoritm

1. An improved enzyme complementation assay comprising
i) adding a substrate to a cell comprising a GPCR-EA fusion protein and a β-arrestin-EB fusion protein, wherein the β-arrestin is mutated,
ii) adding a ligand to obtain, if possible, a GPCR-EA/β-arrestin-EB complex, and
iii) measuring a signal arising from association of EA and EB to create an enzymatically active protein catalyzing conversion of the substrate which leads to a detectable signal,
wherein the improvement leads to an increased signal compared with the signal obtained by use of the same process employing a β arrestin-EB fusion protein, wherein the β-arrestin is wild type β-arrestin.

2. An improved assay according to claim 1, wherein separation of β-arrestin-EB from the GPCR-EA/β-arrestin-EB complex is delayed and/or inhibited.

3. An improved assay according to claim 1 or 2, wherein internalization of the GPCR-EA/β-arrestin-EB complex is inhibited.

4. An improved assay according to claim 1 or 2, wherein β-arrestin is mutated so that its binding to clathrin and/or AP2 is impaired.

5. An improved assay according to claim 4, wherein β-arrestin is further mutated so that it is phosphorylation independent.

6. An improved assay according to claim 1 or 2, wherein β-arrestin is truncated so that it does not contain any clathrin and/or AP2 binding sites.

7. An improved assay according to claim 1 or 2, wherein β-arrestin is mutated by deletion, insertion or substitution so that one or more AP2 binding sites are impaired in their binding to AP2.

8. An improved assay according to claim 1, wherein β-arrestin is specifically mutated so that it acts on the receptor independent of the receptors phosphorylation state.

9. An improved assay according to claim 1 or 2, wherein β-arrestin is originating from an animal source, such as, e.g., from rodents, swine, poultry, cattle, sheep, goats, horses, cats, dogs, monkeys and humans.

10. An improved assay according to claim 1 or 2, wherein β-arrestin is a β-arrestin 1 or β-arrestin 2.

11. An improved assay according to claim 10, wherein the β-arrestin is human β-arrestin 1 374 stop mutant or human β-arrestin 2 373 stop mutant.

12. An improved assay according to claim 10, wherein the β-arrestin is human β-arrestin 2 R393E;R395E mutant.

13. An improved assay according to claim 1 or 2, wherein EA is beta-galactosidase comprising a N-terminal, Δα, deletion, and EB is beta-galactosidase comprising a C-terminal deletion, Δω.

14. An improved assay according to claim 1 or 2, wherein the enzymatically active protein is lactosidase.

15. An improved β-arrestin translocation assay comprising
i) providing a cell expressing a GPCR and comprising a β-arrestin associated with an optically detectable molecule, ODM, wherein the β-arrestin is mutated,
ii) adding a ligand to obtain, if possible, a GPCR/β-arrestin complex, and
iii) detecting a translocation of the optically detectable molecule,
wherein the improvement leads to a increased and prolonged translocation of the β-arrestin associated with an optically detectable molecule as compared with the signal obtained by use of the same assay employing a β-arrestin associated with an optically detectable molecule, wherein the β-arrestin is wild type β-arrestin.

16. An improved assay according to claim 15, wherein separation of β-arrestin-ODM from the GPCR is delayed and/or inhibited.

17. An improved assay according to claim 15, wherein internalization of the GPCR is inhibited.

18. An improved assay according to claim 15 or 16, wherein β-arrestin is mutated so that its binding to clathrin and/or AP2 is impaired.

19. An improved assay according to claim 18, wherein β-arrestin is truncated so that it does not contain any clathrin and/or AP2 binding sites.

20. An improved assay according to claim 18, wherein β-arrestin is mutated by deletion, insertion or substitution so that one or more AP2 binding sites are impaired in their binding to AP2.

21. An improved assay according to claim 15 or 16, wherein β-arrestin is specifically mutated so that it acts on the receptor independent of the receptors phosphorylation state.

22. An improved assay according to claim 15 or 16, , wherein β-arrestin is originating from an animal source, such as, e.g, from rodents, swine, poultry, cattle, sheep, goats, horses, cats, dogs, monkeys and humans.

23. An improved assay according to claim 16 or 16, wherein β-arrestin is a β-arrestin-1 or β-arrestin-2.

24. An improved assay according to claim 23, wherein the β-arrestin is human β-arrestin 1 374 stop mutant or human β-arrestin 2 373 stop mutant.

25. An improved assay according to claim 23, wherein the β-arrestin is human β-arrestin 2 R393E;R395E mutant.

26. An improved assay according to claim 15 or 16, wherein the optically detectable molecule is a fluorescent molecule.

27. An improved assay according to claim 15 or 16, wherein the optically detectable molecule is a luminescent molecule.

28. An improved assay according to claim 26, wherein the fluorescent molecule is a GFP molecule.

29. An improved assay according to claim 15 or 16 for use in drug discovery methods.

30. An improved assay according to claim 1, 2, 15 or 16 for use in high-throughput screening.

31-33. (canceled)

34. The assay of claim 1, 2, 15 or 16 further comprising identifying a GPCR ligand.

35. The assay of claim 34 wherein the ligand is an agonist.

36. The method of claim 34 wherein the ligand is an antagonist.
